(19) United States
(12) Patent Application Publication
(10) Pub. No.: US 2026/0169113 A1
(43) Pub. Date: Jun. 18, 2026

(54) CEST LIPID ARTIFACT ELIMINATION METHOD, MEDIUM, DEVICE, AND MAGNETIC RESONANCE IMAGING APPARATUS

(71) Applicant: ZHEJIANG UNIVERSITY, Zhejiang (CN)

(72) Inventors: Yi ZHANG, Zhejiang (CN); Zhechuan DAI, Zhejiang (CN); Xingwang YONG, Zhejiang (CN)

(73) Assignee: ZHEJIANG UNIVERSITY, Zhejiang (CN)

(21) Appl. No.: 19/389,076

(22) Filed: Nov. 14, 2025

Related U.S. Application Data
(63) Continuation of application No. PCT/CN2024/090878, filed on Apr. 30, 2024.

(30) Foreign Application Priority Data
May 15, 2023 (CN) 202310544289.2

(51) Int. Cl. G01R 33/565; A61B 5/055; G01R 33/48; G01R 33/56

(52) U.S. Cl. CPC G01R 33/56527; A61B 5/055; G01R 33/4828; G01R 33/5602; G01R 33/5608

(57) ABSTRACT

Disclosed are a CEST lipid artifact elimination method, a medium, a device, and a magnetic resonance imaging apparatus. A lipid multi-peak model is incorporated into a simplified water-magnetization transfer (MT) two-pool Bloch-McConnell equation model for iterative solving, and fitting is performed on water, MT, and multi-peak lipid signals to obtain a reference signal for a CEST effect without direct lipid artifacts, and finally, the indirect influence of a lipid signal on a CEST signal is eliminated by normalization. A Bloch-McConnell equation involving lipids can more truly reflect the composition of an acquired signal, so as to obtain a more accurate CEST reference signal. The lipid artifacts in CEST signals can be eliminated without adjusting the collection sequence, thereby improving the accuracy of CEST imaging.

### CROSS-REFERENCE TO RELATED APPLICATION

This application is a continuation of international application of PCT application serial no. PCT/CN2024/090878 filed on Apr. 30, 2024, which claims the priority benefit of China application no. 202310544289.2 filed on May 15, 2023. The entirety of each of the above-mentioned patent applications is hereby incorporated by reference herein and made a part of this specification.

### TECHNICAL FIELD

The present invention belongs to the technical field of magnetic resonance, and particularly relates to lipid artifact elimination and extraction of contrast in chemical exchange saturation transfer (CEST) imaging.

### RELATED ART

Chemical exchange saturation transfer (CEST) imaging in magnetic resonance imaging can provide molecular-level information and reflect different pathological changes. Currently, CEST has considerable applications in the brain, for example, detecting and grading brain gliomas through protein and metabolite information provided by CEST. However, when applied to the body, especially tissues including lipids, such as the breast, CEST is affected by strong lipid artifacts. A method of CEST analysis usually takes the difference between a reference signal and a labeled signal to reflect a CEST effect of a target group. A magnetization transfer ratio asymmetry (MTR_{asym}) analysis, commonly used in the CEST analysis, uses a reference signal acquired at a frequency offset that is symmetric to the target offset relative to the water resonance. This method assumes that the signal of the symmetric frequency includes no CEST effect, and other effects are the same as those in the labeled signal. The difference between the two is the CEST signal. However, in the tissues including lipids, a resonance frequency range of lipids usually overlaps with a frequency range of the reference signal, and the lipid signal is not symmetric about the water resonance frequency, which means that MTR_{asym} may not eliminate lipid influence in the analysis, resulting in lipid artifacts. Additionally, magnetization transfer (MT) may also affect the analysis of MTR_{asym} to a certain extent.

Current lipid artifact elimination methods may be generally divided into (1) an acquisition sequence design method and (2) a post-processing analysis method. The acquisition sequence design method includes a lipid suppression sequence, a water excitation sequence, and a CEST-Dixon water-lipid separation method. These methods all require additional sequence design, which is not conducive to the promotion of clinical applications. The lipid suppression sequence method has a limited effect in suppressing lipids and may have residual lipid signals. The water excitation sequence method increases the energy absorbed by human body, limiting an acquisition rate and an application scope thereof. The CEST-Dixon water-lipid separation method requires data acquisition of multiple times for analysis, resulting in greatly extended collecting time. The post-processing analysis method is mainly a fitting analysis method based on CEST signal normalization. The specific fitting method mainly includes multi-pool Lorentzian fitting, single-pool Lorentzian difference and extrapolated magnetization transfer signal fitting. The single-pool Lorentzian difference and extrapolated magnetization transfer signal fitting do not incorporate lipid signals into the model, resulting in artifacts in the processing. In the method of multi-pool Lorentzian fitting, it is assumed that low-power saturation reaches a steady state, which may not be satisfied in practical applications, and this method has many fitting parameters, requiring large amounts of data for fitting, which causes longer collecting time and lower robustness.

### SUMMARY OF INVENTION

The main objective of the present invention is to overcome the shortcomings of existing methods and provide a post-processing, widely applicable, more comprehensive, and robust chemical exchange saturation transfer (CEST) lipid artifact elimination method based on differential analysis with fitted magnetization transfer and lipid signals (DIGITAL). This method incorporates lipid signals into a model based on Bloch-McConnell equations, obtains reference signals through fitting, and finally performs normalization to eliminate lipid artifacts.

To achieve the aforementioned objectives, the present invention adopts the following technical solutions:

In a first aspect, the present invention provides a CEST lipid artifact elimination method based on a differential analysis with fitted magnetization transfer (MT) and lipid signals, which performs lipid artifact elimination and signal extraction on each voxel of acquired CEST (Chemical Exchange Saturation Transfer) raw data, and steps of processing for each voxel include:
Step 1: linear-interpolation-based offset correction for a main magnetic field frequency B0 is performed on the acquired CEST raw data to obtain a corrected Z-spectrum corresponding to a target voxel;
Step 2: data including only MT, and data comprising a water direct saturation effect and a lipid direct saturation effect in the corrected Z-spectrum is used as fitting samples; parameters of a water-MT-lipid three-pool Bloch-McConnell equation model are fitted within given upper and lower bounds, respectively; the water-MT-lipid three-pool Bloch-McConnell equation model is obtained by incorporating a multi-peak fat Bloch model into a water-MT two-pool Bloch-McConnell equation;
Step 3: fitted model parameters are input into the water-MT-lipid three-pool Bloch-McConnell equation model to generate a CEST reference signal at each frequency point in the corrected Z-spectrum; the acquired signal in the corrected Z-spectrum is subtracted from the CEST reference signal to obtain a CEST effect value corresponding to each frequency point in a CEST effect curve;
Step 4: The CEST effect curve is normalized by dividing it by (1−FF), where FF represents a fat fraction obtained by fitting; in the normalized CEST effect curve, the value at the frequency of interest corresponds to the CEST effect after elimination of lipid artifact interference.

Preferably, the water-MT-lipid three-pool Bloch-McConnell equation model is as follows:

$${\tilde{M}}_{ref} ( t + Δ t ) = exp ( {\tilde{A}}_{w} Δ t ) {\tilde{M}}_{w} ( t ) \cdot ( 1 - FF ) + exp ( {\tilde{A}}_{f} Δ t ) {\tilde{M}}_{f} ( t ) \cdot FF$$

where exp represents an exponential function on a basis of a natural constant e; Δt represents an increment of time t; \tilde{M}_{ref}(t+Δt) is total water-lipid magnetization vector as a reference signal at time t+Δt; \tilde{M}_{w}(t) and \tilde{M}_{f}(t) represent a magnetization vector \tilde{M}_{w} of water and a magnetization vector \tilde{M}_{f} of lipid at time t, respectively;

$${\tilde{M}}_{w} = ( \begin{matrix}{M}_{w} \\ 1\end{matrix} ) , {\tilde{M}}_{f} = ( \begin{matrix}{M}_{f} \\ 1\end{matrix} ) ;$$

in the matrix M_{w}=[M_{xw},M_{yw},M_{zw},M_{zm}]^{T}, M_{xw}, M_{zw}, and M_{zm} represent an x-direction magnetization component of a water pool, a y-direction magnetization component of the water pool, a z-direction magnetization component of water group, and the z-direction magnetization component of MT group, respectively; in the matrix M_{f}=[M_{xf1}, M_{yf1}, M_{zf1}, M_{xf2}, M_{yf2}, M_{zf2}, . . . , M_{xf7}, M_{yf7}, M_{zf7}]^{T}, M_{xf1}, M_{xf2}, . . . , and M_{xf7} represent the x-direction magnetization components of the 1st to 7th lipid groups respectively, M_{yf1}, M_{yf2}, . . . , and M_{yf7} represent the y-direction magnetization components of the 1st to 7th lipid groups respectively, and M_{zf1}, M_{zf2}, . . . , and M_{zf7} represent the z-direction magnetization components of the 1st to 7th lipid groups respectively; the fat fraction FF=M_{0f}/(M_{0f}+M_{0w}) represents a proportion of a lipid signal in a total water-lipid signal, wherein M_{0f} is a sum of the magnetization components M_{0f1}, M_{0f2}, . . . , and M_{0f7} of the 1st to 7th lipid groups under a steady state, and M_{0w} is the magnetization component of the water group under the steady state; in a coefficient matrix

$${\tilde{A}}_{w} = ( \begin{matrix}{A}_{w} & {C}_{w} \\ 0 & 0\end{matrix} ) , {\tilde{A}}_{f} = ( \begin{matrix}{A}_{f} & {C}_{f} \\ 0 & 0\end{matrix} ) ,$$

matrix

$${A}_{w} = ( \begin{matrix}- {R}_{2 w} & - Δ {ω}_{w} & 0 & 0 \\ Δ {ω}_{w} & - {R}_{2 w} & - {ω}_{1} & 0 \\ 0 & {ω}_{1} & - {R}_{1 w} - {k}_{wm} & {k}_{mw} \\ 0 & 0 & {k}_{wm} & - {R}_{1 m} - {R}_{rfm} - {k}_{mv}\end{matrix} )$$

and vector C_{w}=[0,0, R_{1w}M_{0w}, R₁ₘM₀ₘ]^{T}; Δω_{w}=ω_{RF}−ω_{w}−ΔB₀, ω_{RF} represents frequency of an applied radio frequency pulse, ω_{w} represents a resonance frequency of the water group, ΔB₀ represents a residual main magnetic field inhomogeneity to be corrected, ω₁ represents an intensity of the applied radio frequency pulse, k_{wm} and k_{mw} represent exchange rates from the water group to the MT group and from the MT group to the water group, R_{rfm} represents an absorption rate of the MT group to the radio frequency pulse, R_{1w}=1/T_{1w}, R_{2w}=1/T_{2w}, R₁ₘ=1/T₁ₘ, R₂ₘ=1/T₂ₘ represent a longitudinal relaxation rate of the water group, a transverse relaxation rate of the water group, a longitudinal relaxation rate of the MT group, and a transverse relaxation rate of the MT group, respectively; matrix

$${A}_{f} = ( \begin{matrix}{A}_{f 1} & & & \\ & {A}_{f 2} & & \\ & & ⋱ & \\ & & & {A}_{f 7}\end{matrix} )$$

and vector C_{f}=[0, 0, R_{1f1}M_{0f1}, 0, 0, R_{1f2}M_{0f2}, . . . , 0, 0, R_{1f7}M_{0f7}]^{T}, where an nth matrix

$${A}_{fn} = ( \begin{matrix}- {R}_{2 fn} & - Δ {ω}_{fn} & 0 \\ Δ {w}_{fn} & - {R}_{2 fn} & - {ω}_{1} \\ 0 & {ω}_{1} & - {R}_{1 fn}\end{matrix} )$$

of a matrix A_{f1}˜A_{f7}, n=1, 2, . . . , to 7; relative amplitude values M_{0f1}, M_{0f2}, . . . , and M_{0f7} of the seven lipid groups are estimated by three parameters comprising CL, ndb, and nmidb; the parameter CL represents fatty acid chain length, the parameter ndb represents a number of double bonds per molecule, and the nmidb represents a number of methylene double bonds; Δω_{fn}=ω_{RF}−ω_{fn}−ΔB₀, where ω_{fn} represents a resonance frequency of the nth group; R_{1fn}=1/T_{1fn} and R_{2fn}=1/T_{2fn} represent a longitudinal relaxation rate and a transverse relaxation rate of the nth lipid group respectively; longitudinal relaxation times of the seven lipid groups are set to be identical: T_{1f}=T_{1f1}=T_{1f2}= . . . =T_{1f7}, and transverse relaxation times of the seven lipid groups are set to be identical: T_{2f}=T_{2f1}=T_{2f2}= . . . =T_{2f7};

The parameters to be fitted in the model are: T_{1w}, T_{2w}, T₂ₘ, M₀ₘ, k_{wm}, FF, T_{1f}, T_{2f}, ΔB₀, CL, ndb, nmidb.

Preferably, the longitudinal relaxation rate R₁ₘ of the MT group and a resonance frequency of lipid peaks are both input as preset values of prior conditions into the model;

Preferably, the intensity of the applied radio frequency pulse ω₁ is calculated from additionally acquired radio frequency field map and then input as a prior condition into the model.

Preferably, in the Step 2, the data including only MT effect are data points with frequencies preferably in a range from 80 ppm to 6 ppm in the corrected Z-spectrum; the data including the water direct saturation effect are data points with frequencies preferably in a range from 1.5 ppm to −1.5 ppm in the corrected Z-spectrum; the data including the lipid direct saturation effect are data points with frequencies preferably in a range from −1 ppm to −6 ppm in the corrected Z-spectrum;

Preferably, in the Step 2, the data used as fitting samples are data points with frequencies in the ranges from 80 ppm to 10 ppm and 0.75 ppm to −6 ppm in the corrected Z-spectrum.

Preferably, in the Step 2, the parameters to be fitted in the model T_{1w}, T_{2w}, T₂ₘ, M₀ₘ, k_{wm}, FF, T_{1f}, T_{2f}, ΔB₀, CL, ndb, nmidb have upper bounds of [1.50, 0.100, 5e-6, 0.4500, 40, 1, 0.400, 0.150, 64, 18, 5.5, 2.5] respectively, lower bounds of [1.35, 0.015, 1e-6, 0.0001, 20, 0, 0.250, 0.025, −64, 17, 1, 0] respectively during fitting, and initial values [1.35, 0.015, 1e-6, 0.0001, 20, 1, 0.400, 0.025, 0, 17, 1, 0] respectively for fitting;

Preferably, in Step 2, the absorption rate of the MT group to the radio frequency pulse is

$${R}_{r f b} = {ω}_{1}^{2} π g ( 2 π Δ {ω}_{w} ) ;$$

where g(⋅) is a lineshape function, including Gaussian, Lorentzian, and super-Lorentzian; and the lineshape function is preferably super-Lorentzian in breast data.

Preferably, in the Step 2, the data in the range from 80 ppm to 10 ppm is set with a higher weight during fitting, and the weight is 2 times a weight of the data in the range from 0.75 ppm to −6 ppm.

Preferably, in the Step 4, the frequency of interest is 3.5 ppm, and a CEST effect value of 3.5 ppm of the normalized CEST effect curve is an APT^{#} value.

In a second aspect, the present invention provides a computer-readable storage medium. A computer program is stored on the storage medium, and when the computer program is executed by a processor, the DIGITAL-based CEST lipid artifact elimination method according to any one of the preceding contents is implemented.

In a third aspect, the present invention provides a computer electronic device including a processor and a storage medium. A computer program is stored on the storage medium. When the computer program is executed by the processor, the DIGITAL-based CEST lipid artifact elimination method according to any one of the preceding contents is implemented.

In a fourth aspect, the present invention provides a magnetic resonance imaging apparatus configured to eliminate CEST lipid artifacts, which includes a magnetic resonance scanner and a control unit. The magnetic resonance scanner is configured to obtain a CEST image through magnetic resonance CEST imaging. The control unit obtains the CEST image and stores a computer program. When the computer program is executed to implement the DIGITAL-based CEST lipid artifact elimination method according to any one of the preceding contents, the CEST effect value with lipid artifacts eliminated for each voxel is output.

### BRIEF DESCRIPTION OF THE DRAWINGS

FIG. 1 is a schematic diagram of a specific implementation process of the present invention;

FIG. 2 shows acquired data curves and fitted data curves in an embodiment;

FIG. 3 shows a dynamic enhancement image acquired and an APT^{#} image obtained through a method in an embodiment;

FIG. 4 is a fat fraction diagram obtained by fitting using a method proposed by the present invention in an embodiment.

### DESCRIPTION OF THE EMBODIMENTS

The present invention is further described below in combination with specific embodiments and accompanying drawings thereof.

The present invention provides a post-processing, widely applicable, more comprehensive, and robust CEST lipid artifact elimination method based on a differential analysis with fitted magnetization transfer and lipid signals (DIGITAL). The present invention incorporates a seven lipid resonance peak model into a simplified water-magnetization transfer (MT) two-pool Bloch-McConnell equation model, obtains a reference signal for a CEST effect without direct lipid artifacts by fitting signals of water, MT, and lipid, and finally, a scaling effect of a lipid signal on a CEST signal is eliminated by normalization.

A core of the present method lies in reasonably incorporating lipid into the simplified water-MT two-pool Bloch-McConnell equation model, and establishing a water-MT-lipid three-pool Bloch-McConnell equation model. At the same time, reasonable simplification is performed on this model to reduce requirements of a calculation resource of the present method, thereby expanding an application scope of the present method. The water-MT-lipid three-pool Bloch-McConnell equation of the present method is further described below:

The commonly used simplified water-MT two-pool (water pool w and MT pool m) Bloch-McConnell equation in the field may be represented as:

$$\frac{d {M}_{x w}}{d t} = - Δ {ω}_{w} {M}_{y w} - {R}_{2 w} {M}_{x w}$$

$$\frac{d {M}_{y w}}{d t} = Δ {ω}_{w} {M}_{x w} - {R}_{2 w} {M}_{y w} - {ω}_{1} ( t ) {M}_{z w}$$

$$\frac{d {M}_{z w}}{d t} = {ω}_{1} ( t ) {M}_{y w} - {R}_{1 w} ( {M}_{z w} - {M}_{0 w} ) + {k}_{m w} {M}_{z m} - {k}_{w m} {M}_{z w}$$

$$\frac{d {M}_{z m}}{d t} = - {R}_{rfb} {M}_{z m} - {R}_{1 m} ( {M}_{z m} - {M}_{0 m} ) + {k}_{w m} {M}_{z w} - {k}_{m w} {M}_{z m}$$

where M_{xw}, M_{yw}, M_{zw}, and M_{zm} represent an x-direction magnetization component of the water pool, a y-direction magnetization component of the water pool, a z-direction magnetization component of a water group, and the z-direction magnetization component of MT group, respectively; Δω_{w}=ω_{RF}−ω_{w}, ω_{RF} represents frequency of an applied radio frequency pulse, ω_{w} represents a resonance frequency of the water group, ω₁(t) represents an intensity of the radio frequency pulse applied at time t, k_{wm} and k_{mw} represent exchange rates from the water group to the MT group and from the MT group to the water group, respectively, and R_{rfm} represents an absorption rate of the MT group to the radio frequency pulse; R_{1w}=1/T_{1w}, R_{2w}=1/T_{2w}, R₁ₘ=1/T₁ₘ, and R₂ₘ=1/T₂ₘ represent a longitudinal relaxation rate of the water group, a transverse relaxation rate of the water group, a longitudinal relaxation rate of the MT group, and a transverse relaxation rate of the MT group, respectively; M_{0w} and M₀ₘ represent the magnetization of the water group under a steady state and the magnetization of the MT group under a steady state, respectively.

An absorption rate R_{rfb} of the MT group to the radio frequency pulse may be represented as

$${R}_{rfb} = {ω}_{1}^{2} π g ( 2 π Δ {ω}_{w} ) ,$$

where g(⋅) is a lineshape function, including Gaussian, Lorentzian, and super-Lorentzian. Currently, it is generally considered in the field that the super-Lorentzian linear function is closer to MT signal in human body.

For the super-Lorentzian linear function:

$$g ( 2 π Δ {ω}_{m} ) = {\int }_{0}^{π / 2} d θ sin θ \sqrt{\frac{2}{π}} \frac{{T}_{2 m}}{( 3 {cos}^{2} θ - 1 )} {e}^{- 2 {( \frac{2 π Δ {ω}_{m} {T}_{2 m}}{3 {cos}^{2} θ - 1} )}^{2}} ,$$

where Δωₘ=ω_{RF}−ωₘωₘ, and ωₘ represents the resonance frequency of the water group.

The aforementioned simplified water-MT two-pool Bloch-McConnell equation can only describe an exchange process between water signal and the MT signal. When the lipid signal exists, a CEST data curve may contain lipid resonance peaks. During the process of fitting the two-pool, the lipid signal will exist in the fitted data, resulting in a deviation in a result of fitting the two-pool. Additionally, the preprocessing of the CEST data may also be affected by lipid. The CEST signal may be distorted due to modulation by fat content.

One fat resonance peak signal may be described by the Bloch equation:

$$\frac{d {M}_{x f}}{d t} = - Δ {ω}_{f} {M}_{y f} - {R}_{2 f} {M}_{x f}$$

$$\frac{d {M}_{y f}}{d t} = Δ {ω}_{f} {M}_{x f} - {R}_{2 f} {M}_{y f} - {ω}_{1} ( t ) {M}_{z f}$$

$$\frac{d {M}_{z f}}{d t} = {ω}_{1} ( t ) {M}_{y f} - {R}_{1 f} ( {M}_{z f} - {M}_{0 f} )$$

where M_{xf}, M_{yf}, M_{zf} represent the x-, y-, z-direction magnetization components respectively; Δω_{f}=ω_{RF}−ω_{f}, ω_{RF} represents the frequency of the applied radio frequency pulse, ω_{f} represents a resonance frequency of a resonance peak, ω₁(t) represents an intensity of the radio frequency pulse applied at time t, R_{1f}=1/T_{1f} and R_{2f}=1/T_{2f} represent a longitudinal relaxation rate and a transverse relaxation rate respectively, and M_{0f} represents a magnetization under the steady state.

For the water-MT two-pool Bloch-McConnell equation, a matrix format is written as follows:

$$\frac{d {M}_{w}}{d t} = {A}_{w} {M}_{w} + {C}_{w}$$

where M_{w}=[M_{xw}, M_{yw}, M_{zw}, M_{zm}]^{T}

$${A}_{w} = ( \begin{matrix}- {R}_{2 w} & - Δ {ω}_{w} & 0 & 0 \\ Δ {ω}_{w} & - {R}_{2 w} & - {ω}_{1} & 0 \\ 0 & {ω}_{1} & - {R}_{1 w} - {k}_{wm} & {k}_{mw} \\ 0 & 0 & {k}_{wm} & - {R}_{1 m} - {R}_{rfm} - {k}_{mv}\end{matrix} ) ,$$

$${C}_{w} = {[ 0 , 0 , {R}_{1 w} {M}_{0 w} , {R}_{1 m} {M}_{0 m} ]}^{T} .$$

Further simplification yields:

$$\frac{d {\tilde{M}}_{w}}{d t} = {\tilde{A}}_{w} {\tilde{M}}_{w}$$

where

$${\tilde{A}}_{w} = ( \begin{matrix}{A}_{w} & {C}_{w} \\ 0 & 0\end{matrix} )$$

$${\tilde{M}}_{w} = ( \begin{matrix}{M}_{w} \\ 1\end{matrix} ) .$$

The solution to the aforementioned equation is:

$${\tilde{M}}_{w} ( t + Δ t ) = exp ( {\tilde{A}}_{w} Δ t ) {\tilde{M}}_{w} ( t )$$

#(1)

where \tilde{M}_{w}(t+Δt) is a magnetization vector of the water-MT two-pool Bloch-McConnell equation model at a moment (t+Δt).

Similarly, for a single peak of lipid, after the Bloch equation is simplified and solved according to the aforementioned method, the solution is:

$${\tilde{M}}_{f}^{1} ( t + Δ t ) = exp ( {\tilde{A}}_{f}^{1} Δ t ) {\tilde{M}}_{f}^{1} ( t )$$

#(2)

where

$${\tilde{M}}_{f}^{1} ( t + Δ t )$$

is a magnetization vector of a single resonance peak of lipid at a moment (t+Δt) and

$${\tilde{A}}_{f}^{1} = ( \begin{matrix}{A}_{f}^{1} & {C}_{f}^{1} \\ 0 & 0\end{matrix} )$$

$${\tilde{M}}_{f}^{1} = ( \begin{matrix}{M}_{f}^{1} \\ 1\end{matrix} )$$

$${A}_{f}^{1} = ( \begin{matrix}- {R}_{2 f}^{1} & - {Δω}_{f}^{1} & 0 \\ Δ {ω}_{f}^{1} & - {R}_{2 f}^{1} & - {ω}_{1}^{1} \\ 0 & {ω}_{1}^{1} & - {R}_{1 f}^{1}\end{matrix} )$$

$${C}_{f}^{1} = {[ 0 , 0 , {R}_{1 f}^{1} {M}_{0 f}^{1} ]}^{T} .$$

The superscript “1” in the equation represents the lipid signal of 1st resonance peak.

Since there is no chemical exchange or other interactions between the various lipid resonance peaks, a lipid multi-peak signal may be obtained by summing multiple lipid peak signals individually according to a relative amplitude of lipid peaks. The summing process may be completed by matrix operations, making

$${\tilde{A}}_{f} = ( \begin{matrix}{A}_{f} & {C}_{f} \\ 0 & 0\end{matrix} )$$

$${\tilde{M}}_{f} = ( \begin{matrix}{M}_{f} \\ 1\end{matrix} )$$

where a matrix

$${A}_{f} = ( \begin{matrix}{A}_{f 1} & & & \\ & {A}_{f 2} & & \\ & & ⋱ & \\ & & & {A}_{f 7}\end{matrix} ) ,$$

and a vector C_{f}=[0, 0, R_{1f1}M_{0f1}, 0, 0, R_{1f2}M_{0f2}, . . . , 0, 0, R_{1f7}M_{0f7}]^{T}, and an nth matrix

$${A}_{f n} = ( \begin{matrix}- {R}_{2 fn} & - {Δω}_{fn} & 0 \\ {Δω}_{fn} & - {R}_{2 fn} & - {ω}_{1} \\ 0 & {ω}_{1} & - {R}_{1 fn}\end{matrix} )$$

of a matrix A_{f1}˜A_{f7}. Relative amplitude M_{0f1}, M_{0f2}, . . . , and M_{0f7} of the seven lipid groups may differ in a tissue, and the relationships between the relative amplitude values may be estimated by three parameters. These parameters include a fatty acid chain length (CL), a number of double bonds per molecule (ndb), and a number of methylene double bonds (nmidb). Specifically, in each fat molecule, the hydrogen proton density contained in each of the seven groups may be represented by the aforementioned three parameters as:

#1 group includes hydrogen proton density M₁=9.

#2 group includes hydrogen proton density M₂=((CL−4)×6)−(ndb×8)+(nmidb×2).

#3 group includes hydrogen proton density M₃=6.

#4 group includes hydrogen proton density M₄=6+(ndb−nmidb)×4.

#5 group includes hydrogen proton density M₅=nmidb×2.

#6 group includes hydrogen proton density M₆=4.

#7 group includes hydrogen proton density M₇=ndb×2+1.

The relationships between the relative amplitudes of the aforementioned seven lipid groups are determined by lipid chemical compositions. By dividing the hydrogen proton density Mₙ of any nth group by the total hydrogen proton density included in one lipid molecule, the relative amplitude values between the groups may be obtained, where n=1, 2, . . . , to 7.

Δω_{fn}=ω_{RF}−ω_{fn}−ΔB₀, where ω_{fn} represents a resonance frequency of the nth group; R_{1fn}=1/T_{1fn} and R_{2fn}=1/T_{2fn} represent the longitudinal relaxation rate and the transverse relaxation rate of the nth lipid group respectively.

In M_{f}=[M_{xf1}, M_{yf1}, M_{zf1}, M_{xf2}, M_{yf2}, M_{zf2}, . . . , M_{xf7}, M_{yf7}, M_{zf7}]^{T}, M_{xf1}, M_{xf2} . . . , and M_{xf7} are x-direction magnetization components of 1st to 7th lipid groups respectively; M_{yf1}, M_{yf2}, . . . , and M_{yf7} are y-direction magnetization components of the 1st to 7th lipid groups respectively; M_{zf1}, M_{zf2}, . . . , and M_{zf7} are z-direction magnetization components of the 1st to 7th lipid groups respectively.

Through the aforementioned changes, a sum of the multi-peak lipid signals may be represented as:

$${\tilde{M}}_{f} ( t + Δ t ) = exp ( {\tilde{A}}_{f} Δ t ) {\tilde{M}}_{f} ( t )$$

#(3)

Through Equation (1) and Equation (3), the water signal M_{w}(ω_{RF}) and the lipid signal M_{f}(ω_{RF}) after saturation by a specific saturation pulse frequency a)_{RF} may be iteratively calculated respectively. There is no chemical exchange between water and lipid, and finally, the acquired signal may be simply represented as a sum of the water signal and the lipid signal:

$${M}_{a c q} ( {ω}_{R F} ) = {M}_{w} ( {ω}_{R F} ) + {M}_{f} ( {ω}_{R F} ) .$$

When CEST data are processed, the acquired data needs to be normalized by unsaturated image data to calculate a Z-spectrum. The reference Z-spectrum M_{ref} may be obtained by dividing fitted signal by unsaturated data:

$${M}_{r e f} ( {ω}_{R F} ) = \frac{{M}_{a c q} ( {ω}_{R F} )}{{M}_{0}} = \frac{{M}_{w} ( {ω}_{R F} ) + {M}_{f} ( {ω}_{R F} )}{{M}_{0 w} + {M}_{0 f}}$$

Let the fat fraction FF be a proportion of an unsaturated lipid to an unsaturated water-lipid signal, FF=M_{0f}/(M_{0f}+M_{0w}), the aforementioned equation may be simplified as:

$${M}_{r e f} ( {ω}_{R F} ) = \frac{{M}_{w} ( {ω}_{R F} )}{{M}_{0 w}} \cdot ( 1 - FF ) + \frac{{M}_{f} ( {ω}_{R F} )}{{M}_{0 f}} \cdot FF$$

In the aforementioned equation, M_{0w} and M_{0f} may be eliminated by setting the steady state value to 1 during fitting. It should be noted that the steady state value during fitting does not need to be equal to the steady state value in the fat fraction. In this way, the aforementioned equation may be further simplified to:

$${M}_{r e f} ( {ω}_{R F} ) = {M}_{w} ( {ω}_{R F} ) \cdot ( 1 - FF ) + {M}_{f} ( {ω}_{R F} ) \cdot FF$$

#(4)

In this way, it is equivalent to directly fitting a percentage that a saturated water and lipid signals account for of the unsaturated signals, that is, directly fitting the Z-spectrum, and then adding according to a ratio of the unsaturated water and lipid signals.

From equations (1), (3), and (4), the equations for the reference signal varying with time may be obtained as:

$${\tilde{M}}_{r e f} ( t + Δ t ) = exp ( {\tilde{A}}_{w} Δ t ) {\tilde{M}}_{w} ( t ) \cdot ( 1 - FF ) + exp ( {\tilde{A}}_{f} Δ t ) {\tilde{M}}_{f} ( t ) \cdot FF$$

#(5)

Considering the CEST effect at a specific frequency of interest ωᵢ, a CEST effect value CEST(ωᵢ) calculated by the reference signal may be represented as:

$$CEST ( {ω}_{i} ) = {M}_{ref} ( {ω}_{i} ) - {M}_{acq} ( {ω}_{i} )$$

where M_{acq}(ωᵢ) is the data of the Z-spectrum at a saturation frequency co. Since the CEST effect is generated due to exchange between solute molecules and water, the lipid signals are the same in the reference signal and the acquired signal.

Therefore:

$$CEST ( {ω}_{i} ) = ( {M}_{wref} - {M}_{wacq} ) \cdot ( 1 - FF )$$

where M_{wref} and M_{wacq} represent the water signals in the reference data and acquired data respectively. According to the aforementioned equation, the directly calculated CEST effect is affected by (1−FF). Dividing both sides of the aforementioned equation by (1−FF) may eliminate the scaling of the fat fraction on the CEST effect. The CEST effect corrected by the fat fraction is denoted as CEST′(ωᵢ), which may be:

$${CEST}^{′} ( {ω}_{i} ) = \frac{CEST ( {ω}_{i} )}{1 - FF} = \frac{{M}_{ref} ( {ω}_{i} ) - {M}_{acq} ( {ω}_{i} )}{1 - FF}$$

#(6)

Equation (5) is a model used in the present invention. Equation (6) is an algorithm to calculate the CEST effect using this model.

Due to relatively close relaxation rates of lipid, it is assumed that T_{1f}=T_{1f1}=T_{1f2}=. . . =T_{1f7} and T_{2f}=T_{2f1}=T_{2f2}= . . . =T_{2f7} to reduce fitting computation. A longitudinal relaxation rate R₁ₘ of MT and the resonance frequency of the lipid peaks are input into the model as prior conditions according to data from literature. Finally, the model of the present invention includes 12 unknown parameters, which are T_{1w}, T_{2w}, T₂ₘ, M₀ₘ, k_{wm}, FF, T_{1f}, T_{2f}, ΔB₀, CL, ndb, nmidb, and the remaining parameters are all input as a prior condition into the model. Through fitting the model, a reference signal including lipid and an MT effect may be obtained to eliminate the direct lipid influence on the CEST effect, and then the CEST effect is renormalized according to a CEST effect calculation equation to eliminate the scaling effect of lipids on the CEST, so that the CEST effect without lipid artifact can be obtained.

Therefore, according to the aforementioned theoretical description, the CEST lipid artifact elimination method based on the DIGITAL in the present invention may be specifically described as follows:

A computer device or a control unit with data processing capability first reads raw data (that is, CEST images) acquired by a magnetic resonance imaging apparatus through magnetic resonance CEST imaging. The reading manner may be online real-time reading from the magnetic resonance imaging apparatus, or the raw data may first be acquired by the magnetic resonance imaging apparatus and stored in a data storage device, and then read offline from the data storage device. Lipid artifact elimination and signal extraction are performed on each voxel in the acquired CEST raw data. The process for each voxel includes:

Step 1: Linear interpolation-based offset correction of main magnetic field frequency B0 is performed on the acquired CEST raw data to obtain a corrected Z-spectrum corresponding to a target voxel.

Step 2: In the corrected Z-spectrum, the data including MT as well as the data including a water direct saturation effect and a lipid direct saturation effect are used as fitting samples, The model parameters of the water-MT-lipid three-pool Bloch-McConnell equation model are fitted in given upper and lower bounds. The water-MT-lipid three-pool Bloch-McConnell equation model is as described in Equation (5), which is not repeated here.

It should be noted that in the process of extracting data points from the corrected Z-spectrum as fitting samples, the specific selection of which frequency ranges of data is determined based on the frequency ranges where the three effects are located. Generally, the data including MT effect are data points with frequencies in a range from 80 ppm to 6 ppm in the corrected Z-spectrum; the data including the water direct saturation effect are data points with frequencies in a range from 1.5 ppm to −1.5 ppm in the corrected Z-spectrum; and the data including the lipid direct saturation effect are data points with frequencies in a range from −1 ppm to −6 ppm in the corrected Z-spectrum. Theoretically, the data points within these three frequency ranges extracted from the corrected Z-spectrum may serve as fitting samples. However, considering the fitting accuracy, the frequencies of the data points should preferably not fall around the frequency of interest. For example, if the frequency of interest is selected as 3.5 ppm, data points near 3.5 ppm should preferably not be used as fitting samples. Therefore, it is preferred to set the data used as fitting samples to be data in the corrected Z-spectrum with frequencies in the ranges from 80 ppm to 10 ppm and 0.75 ppm to −6 ppm.

Step 3: The fitted model parameters are input into the water-MT-lipid three-pool Bloch-McConnell equation model to generate the CEST reference signal of each frequency point in the corrected Z-spectrum. The actual acquired signal in the corrected Z-spectrum is subtracted from the reference signal corresponding to each frequency point to obtain the CEST effect value of the CEST effect curve corresponding to frequency point.

Step 4: The CEST effect curve is normalized by dividing by (1−FF), where FF is the fat fraction obtained by fitting; in the normalized CEST effect curve, the value of the frequency of interest is the CEST effect value after lipid artifact is eliminated.

It should be noted that the selection of the frequency of interest is arbitrary, but in this art, the selected frequency of interest usually is the resonance frequency of the group including the CEST effect, to reflect the signal of the group at the frequency of interest. For example, when the frequency of interest is selected as 3.5 ppm, the CEST effect value calculated by the present method is APT^{#} (Amide proton transfer signal from DIGITAL analysis; amide proton transfer signal excluding lipid artifacts obtained by the CEST lipid artifact elimination method based on the DIGITAL) value. Other frequencies that may reflect the CEST effect may also be selected as the frequency of interest, and the present method does not limit the selection of the frequency of interest.

Below, the present application provides further description of the method proposed by the present invention through the following non-limiting embodiments:

### Embodiment

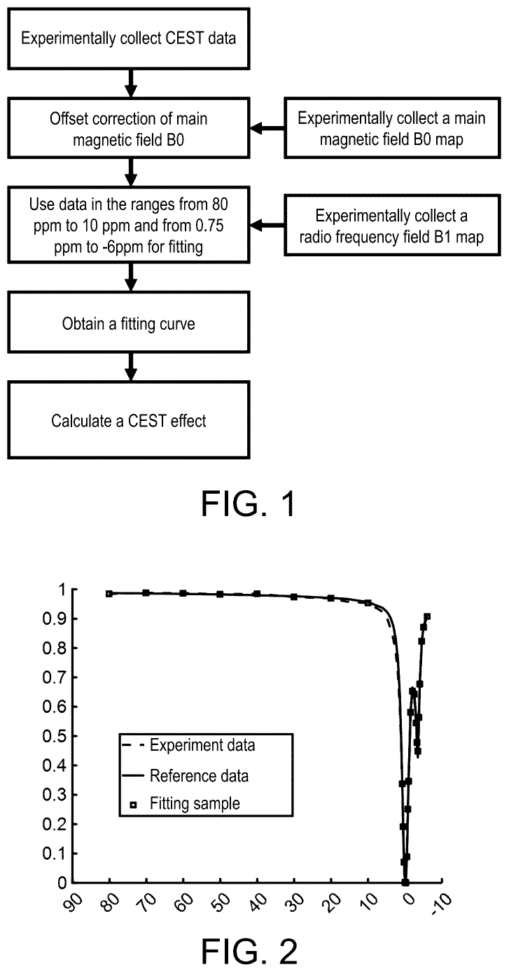

In this embodiment, the specific implementation process of the DIGITAL method is shown in FIG. 1. The method proposed by the present invention requires to first collect CEST image data, and perform the DIGITAL method on each voxel of the acquired CEST image data, voxel by voxel, that is, sequentially executing Step 1 to Step 4, to calculate and obtain the APT^{#} value with lipid artifact eliminated for each voxel. The specific implementation process of Step 1 to Step 4 are described in detail below.

Step 1: The linear interpolation-based offset correction of the main magnetic field frequency B0 is performed on the acquired CEST raw data to obtain a corrected Z-spectrum corresponding to the target voxel.

Step 2: In the corrected Z-spectrum, the data including MT, the water direct saturation effect and the lipid direct saturation effect are used as fitting samples. The established water-MT-lipid three-pool Bloch-McConnell equation model is used for fitting according to given upper and lower bounds, where the model is used to calculate the reference signal of the Z-spectrum. As described above, the water-MT-lipid three-pool Bloch-McConnell equation model used by the present method is as follows:

$${\tilde{M}}_{ref} ( t + Δ t ) = exp ( {\tilde{A}}_{w} Δ t ) {\tilde{M}}_{w} ( t ) \cdot ( 1 - FF ) + exp ( {\tilde{A}}_{f} Δ t ) {\tilde{M}}_{f} ( t ) \cdot FF$$

where exp represents an exponential function on the basis of a natural constant e; Δt represents an increment of time t; \tilde{M}_{ref}(t+Δt) is the total water-lipid magnetization vector as a reference signal at time t+Δt; \tilde{M}_{w}(t) and \tilde{M}_{f}(t) represent a magnetization vector \tilde{M}_{w} of water and a magnetization vector \tilde{M}_{f} of lipid at time t respectively;

$${\tilde{M}}_{w} = ( \begin{matrix}{M}_{w} \\ 1\end{matrix} ) ‵ {\tilde{M}}_{f} = ( \begin{matrix}{M}_{f} \\ 1\end{matrix} ) ;$$

in the matrix M_{w}=[M_{xw}, M_{yw}, M_{zw}, M_{zm}]^{T}, M_{xw}, M_{yw}, M_{zw}, and M_{zm} represent the x-direction magnetization component of the water pool, the y-direction magnetization component of the water pool, the z-direction magnetization component of the water group, and the z-direction magnetization component of the MT group, respectively; in the matrix M_{f}=[M_{xf1}, M_{yf1}, M_{zf1}, M_{xf2}, M_{yf2}, M_{zf2}, . . . , M_{xf7}, M_{yf7}, M_{zf7}]^{T}, M_{xf1}, M_{xf2}, . . . , and M_{xf7} represent the x-direction magnetization components of the 1st to 7th lipid groups respectively, M_{yf1}, M_{yf2}, . . . , and M_{yf7} represent the y-direction magnetization components of the 1st to 7th lipid groups respectively, and M_{zf1}, M_{zf2}, . . . , and M_{zf7} represent the z-direction magnetization components of the 1st to 7th lipid groups respectively; fat fraction FF=M_{0f}/(M_{0f}+M_{0w}) represents the proportion of the lipid signal in a total water-lipid signal, where M_{0f} is a sum of the magnetization components M_{0f1}, M_{0f2}, . . . , and M_{0f7} of the 1st to 7th lipid groups under the steady state, and M_{0w} is the magnetization component of the water group under the steady state; coefficient matrix

$${\tilde{A}}_{w} = ( \begin{matrix}{A}_{w} & {C}_{w} \\ 0 & 0\end{matrix} ) , {\tilde{A}}_{f} = ( \begin{matrix}{A}_{f} & {C}_{f} \\ 0 & 0\end{matrix} ) ,$$

matrix

$$\begin{matrix}{A}_{w} = \\ ( \begin{matrix}- {R}_{2 w} & - Δ {ω}_{w} & 0 & 0 \\ {Δω}_{w} & - {R}_{2 w} & - {ω}_{1} & 0 \\ 0 & {ω}_{1} & - {R}_{1 w} - {k}_{wm} & {k}_{mw} \\ 0 & 0 & {k}_{wm} & - {R}_{1 m} - {R}_{rfm} - {k}_{mw}\end{matrix} )\end{matrix}$$

and vector C_{w}=[0,0, R_{1w}M_{0w}, R₁ₘM₀ₘ]^{T}; Δω_{w}=ω_{RF}−ω_{w}−ΔB₀, ω_{RF} represents the applied radio frequency pulse frequency, ω_{w}, represents the resonance frequency of the water group, ΔB₀ represents the residual main magnetic field inhomogeneity to be corrected, ω₁ represents the intensity of the applied radio frequency pulse, k_{wm} and k_{mw} represent the exchange rates from the water group to the MT group and from the MT group to the water group. R_{rfb}=ω₁²πg(2πω_{b}), where g(⋅) is super-Lorentzian type, representing the absorption rate of the MT group for radio frequency pulses; R_{1w}=1/T_{1w}, R_{2w}=1/T_{2w}, R₁ₘ=1/T₁ₘ, and R₂ₘ=1/T₂ₘ represent the longitudinal relaxation rate and the transverse relaxation rate of the water group, the longitudinal relaxation rate and the transverse relaxation rate of the MT group, respectively, and M₀ₘ is the magnetization intensity of the MT group under the steady state; matrix

$${A}_{f} = ( \begin{matrix}{A}_{f 1} & & & \\ & {A}_{f 2} & & \\ & & ⋱ & \\ & & & {A}_{f 7}\end{matrix} )$$

and vector C_{f}=[0, 0, R_{1f1}M_{0f1}, 0, 0, R_{1f2}M_{0f2}, . . . , 0, 0, R_{1f7}M_{0f7}]^{T}, where the nth matrix

$$\begin{matrix}{A}_{f} = \\ {A}_{f} = ( \begin{matrix}- {R}_{2 fn} & - Δ {ω}_{fn} & 0 \\ {Δω}_{fn} & - {R}_{2 fn} & - {ω}_{1} \\ 0 & {ω}_{1} & - {R}_{1 fn}\end{matrix} )\end{matrix}$$

of the matrix A_{f1}˜A_{f7}, n=1, 2, . . . , to 7; The relative amplitude values M_{0f1}, M_{0f2}, . . . , and M_{0f7} of the seven lipid groups may differ in tissues, and the relationships may be estimated through three parameters. These parameters include fatty acid chain length (CL), a number of double bonds per molecule (ndb), and a number of methylene double bonds (nmidb). Specifically, in each fat molecule, the hydrogen proton density contained in each of the seven groups may be represented by using the aforementioned three parameters as:

#1 group includes hydrogen proton density M₁=9.

#2 group includes hydrogen proton density M₂=((CL−4)×6)−(ndb×8)+(nmidb×2).

#3 group includes hydrogen proton density M₃=6.

#4 group includes hydrogen proton density M₄=6+(ndb−nmidb)×4.

#5 group includes hydrogen proton density M₅=nmidb×2.

#6 group includes hydrogen proton density M₆=4.

#7 group includes hydrogen proton density M₇=ndb×2+1.

The relationships between the relative amplitude values of the aforementioned seven lipid groups are determined by the lipid chemical compositions. By dividing the hydrogen proton density Mₙ of any nth group by the total hydrogen proton density included in one lipid molecule, the relative amplitude values between the groups may be obtained, where n=1, 2, . . . , to 7. Δω_{fn}=ω_{RF}−ω_{fn}−ΔB₀, where ω_{fn} represents the resonance frequency of the nth lipid group, and R_{1fn}=1/T_{1fn} and R_{2fn}=1/T_{2fn} represent the longitudinal relaxation rate and the transverse relaxation rate of the nth lipid group respectively.

In the aforementioned model, since lipid includes seven resonance peaks and too many unknown parameters exist in the model, simplification is performed on the model with seven lipid peaks to a certain extent. The main differences of the seven lipid peaks lie in the resonance frequency and relative amplitude values, while the relaxation rates do not have significant differences. Therefore, it is assumed that the relaxation rates of lipid with different resonance peaks are the same in the model, that is, T_{1f}=T_{1f1}=T_{1f2}= . . . =T_{1f7}, T_{2f}=T_{2f1}=T_{2f2}= . . . =T_{2f7}. In addition, this method requires additional collection of radio frequency field maps to calculate the actual radio frequency pulse intensity ω₁ received by each voxel.

After the simplification is performed, the model used by the present method includes 12 undetermined parameters, which are T_{1w}, T_{2w}, T₂ₘ, M₀ₘ, k_{wm}, FF, T_{1f}, T_{2f}, ΔB₀, CL, ndb, nmidb. These undetermined parameters are fitted by selecting data including only MT effect as well as data including the water direct saturation effect and the lipid direct saturation effect in the Z spectrum as samples. In this embodiment, the selected data are data with frequency in the ranges from 80 ppm to 10 ppm and 0.75 ppm to −6 ppm. During fitting, the preference of this embodiment is to set a weight of data in the range of 80 ppm to 10 ppm to be 2 times a weight of data in the range of 0.75 ppm to −6 ppm to balance the difference in the number of the two parts of data.

In this embodiment, 12 undetermined parameters T_{1w}, T_{2w}, T₂ₘ, M₀ₘ, k_{wm}, FF, T_{1f}, T_{2f}, ΔB₀, CL, ndb, nmidb may be directly estimated by fitting the samples to obtain fitted values in the range of upper and lower bounds that minimize the total fitting error of the present model. The total fitting error may be represented by the minimum mean square error, and the weight setting may be achieved by changing the coefficient of the error when the minimum root mean square error is calculated. The specific fitting method may be implemented by using MATLAB, Python, or other existing technologies.

During fitting, the upper and lower ranges of the undetermined parameters T_{1w}, T_{2w}, T₂ₘ, M₀ₘ, k_{wm}, FF, T_{1f}, T_{2f}, ΔB₀, CL, ndb, nmidb may be adjusted according to the fitting data. When different human tissues or water phantoms are fitted, the undetermined parameters need to be adjusted according to the normal parameter range of the tissue or water phantom. In this example, when breast data is fitted, the upper bounds of parameters T_{1w}, T_{2w}, T₂ₘ, M₀ₘ, k_{wm}, FF, T_{1f}, T_{2f}, ΔB₀, CL, ndb, nmidb are set to [1.50, 0.100, 5e-6, 0.4500, 40, 1, 0.400, 0.150, 64, 18, 5.5, 2.5] respectively, the lower bounds are set to [1.35, 0.015, 1e-6, 0.0001, 20, 0, 0.250, 0.025, −64, 17, 1, 0] respectively, and the initial values for fitting are [1.35, 0.015, 1e-6, 0.0001, 20, 1, 0.400, 0.025, 0, 17, 1, 0] respectively.

Step 3: The fitted parameters are input into the model proposed by the present method to calculate a reference curve including the MT effect, the water direct saturation effect, and the lipid direct saturation effect. The abscissa of the curve is the input fitting frequency ω. The ordinate is the z-direction magnetization component of the reference signal \tilde{M}_{ref}. The corrected Z-spectrum curve is subtracted from the reference curve point by point to obtain the CEST effect curve. It should be noted that the corrected Z-spectrum curve is actually formed by discrete data points. In the calculation of the CEST effect curve, the corresponding reference signal should be calculated by substituting the fitted parameters into the model at each frequency point of the Z-spectrum curve. Next, the reference signal should be subtracted from the original value at the corresponding frequency of the Z-spectrum curve, thus obtaining the CEST effect value at the corresponding frequency of the CEST effect curve.

Step 4: The CEST effect curve calculated in step 3 is divided by the fitted (1−FF) for the voxel for renormalization, so as to eliminate the scaling effect of the lipid signal on the CEST effect, and obtain the corrected CEST effect curve, where the value at 3.5 ppm is the APT^{#} effect value.

Of course, in the steps of calculating the reference curve, it is not necessary to calculate the complete reference curve. Only the reference value at 3.5 ppm and the B0-corrected Z-spectrum value at 3.5 ppm need to be calculated to obtain the CEST effect value at 3.5 ppm, and the corrected CEST effect value obtained by dividing by (1−FF) can be used as the APT^{#} effect value.

After Step 1 to Step 4 are performed on all voxels of the CEST image, each voxel corresponds to an APT^{#} value. By mapping these APT^{#} values to their corresponding voxels, an APT^{#} image may be obtained.

The DIGITAL method was applied to a breast tumor patient for testing to further demonstrate the technical effect of the method. In this embodiment, the imaging sequence for collecting CEST data includes a CEST saturation module and a fast spin echo acquisition module. The CEST saturation module including 10 Gaussian lineshape saturation pulses with a duration of 100 ms, an effective amplitude value of 1 uT, and an interval of 10 ms between saturation pulses. It should be noted that the present method does not require lipid suppression during CEST imaging, so there is no need to add a lipid suppression module during CEST acquisition. Additional main magnetic field B0 maps and radio frequency field B1 maps were also acquired for B0 correction and parameter fitting.

As shown in FIG. 1, the acquired CEST Z-spectrum data may be corrected for the main magnetic field B₀ offset according to the acquired main magnetic field B₀ map. The acquired Z-spectrum is shown as experimental data in FIG. 2. Subsequently, DIGITAL fitting may be performed for all voxels, with fitting samples being data with the frequency ranges of 80 ppm to 10 ppm and 0.75 ppm to −6 ppm. The acquired radio frequency field B₁ map may also be input into the fitting process for calculating radio frequency ω₁. The upper and lower bounds as well as initial values of parameters may be the same as in Step 2. The samples used for fitting and the reference curve obtained by fitting are shown in FIG. 2.

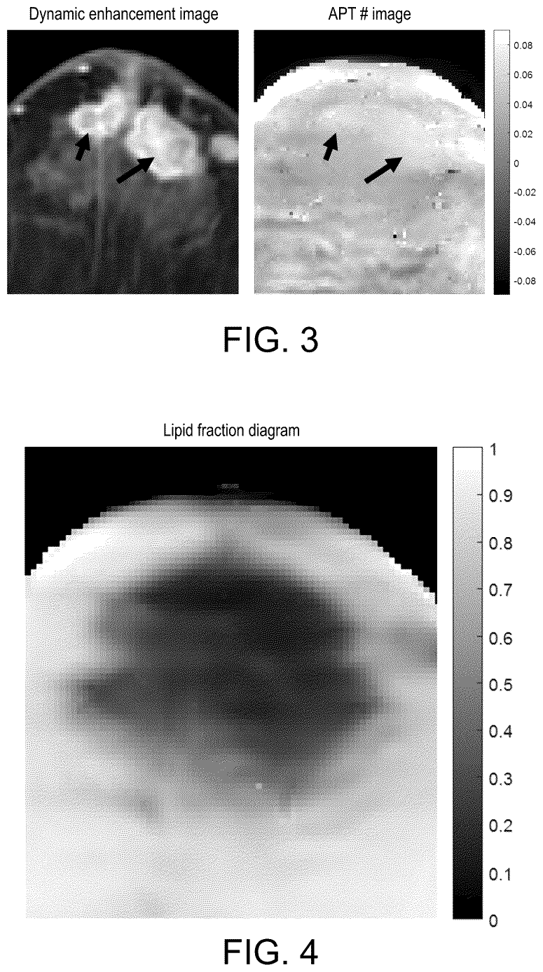

The experimental data obtained in this example are shown in FIG. 3, where the APT^{#} image shows high signal regions corresponding to those of the dynamic contrast enhanced image (indicated by black arrows in FIG. 3), and the color bar on the right side applies only to the APT^{#} image. The fat fraction map obtained in this experiment is shown in FIG. 4, where obvious fat fraction changes may be observed. Comparing the APT^{#} image (FIG. 3) with the fat fraction map (FIG. 4), the APT^{#} effect values of glandular regions with low fat fraction and glandular regions with high fat fraction show no significant difference, suggesting that lipid artifacts have been eliminated.

Similarly, based on the same inventive concept, another preferred embodiment of the present invention also provides a computer electronic device corresponding to the CEST lipid artifact elimination method based on the DIGITAL provided by the aforementioned embodiments, which includes a memory and a processor;
the storage medium is configured to store a computer program;
the processor is configured to implement the CEST lipid artifact elimination method based on the DIGITAL described above when executing the computer program.

Similarly, based on the same inventive concept, another preferred embodiment of the present invention also provides a computer-readable storage medium corresponding to the CEST lipid artifact elimination method based on DIGITAL provided by the aforementioned embodiments. The computer program is stored on the storage medium. When the computer program is executed by a processor, the CEST lipid artifact elimination method based on the DIGITAL described above is implemented.

It may be understood that the storage medium and the memory may include a random access memory (RAM), and may also include a non-volatile memory (NVM), for example, at least one disk memory. Meanwhile, the storage medium may also be various media capable of storing program codes, such as a USB flash drive, a mobile hard drive, a magnetic disk, or an optical disk. Of course, with the widespread application of cloud servers, a software program may also be deployed on a cloud platform to provide corresponding services. Therefore, the computer-readable storage medium is not limited to the local hardware.

It may be understood that the processor may be a general-purpose processor, including a central processing unit (CPU) and a network processor (NP), and may also be a digital signal processor (DSP), an application specific integrated circuit (ASIC), a field-programmable gate array (FPGA), other programmable logic devices, a discrete gate, a transistor logic device, or a discrete hardware component.

Additionally, it should be noted that those skilled in the art may clearly understand that, for convenience and brevity of description, the specific working process of the apparatus described above may refer to the corresponding process in the aforementioned method embodiments, which is not repeated here. In each embodiment provided in the present application, the division of steps or modules in the apparatus and method is merely a logical functional division, and there may be other methods of division in actual implementation, for example, multiple modules or steps may be combined or integrated together, and a single module or step may also be split.

Furthermore, logic instructions of the memory may be implemented in the software functional units and may be stored in a computer-readable storage medium when sold or used as independent products. Based on such understanding, the technical solution of the present invention essentially or the part that contributes to the prior art or the part of the technical solution may be embodied in a software product, the computer software product is stored in a storage medium, including several instructions for making a computer device (which may be a personal computer, a server, or a network device) to execute all or part of the steps of the method described in various embodiments of the present invention.

Similarly, based on the same inventive concept, another preferred embodiment of the present invention also provides a magnetic resonance imaging device corresponding to the CEST lipid artifact elimination method based on DIGITAL provided by the aforementioned embodiment, which includes a magnetic resonance scanner and a control unit;

The magnetic resonance scanner is configured to obtain the CEST images through magnetic resonance CEST imaging;

the control unit may obtain the CEST image and include a computer program stored therein. When the computer program is executed for implementing the CEST lipid artifact elimination method based on the DIGITAL, and the CEST effect value with lipid artifacts eliminated for each voxel is output.

It should be noted that the magnetic resonance imaging device may be any magnetic resonance scanner capable of implementing parallel imaging methods, the structure of which belongs to the prior art and may adopt mature commercial products, with no limitation on specific models. In addition, the control unit of the magnetic resonance imaging device should include the computer program, imaging sequences necessary for implementing CEST imaging, and other software programs.

The modules and functions involved in the aforementioned embodiments of the present invention may be implemented by circuits, other hardware, or executable program codes, as long as the corresponding functions may be achieved. If the code is adopted, the code may be stored in the storage device and executed by corresponding elements in the computer device. The implementation of the present invention is not limited to any specific combination of hardware and software. Each hardware model in the present invention may adopt commercially available products, which may be selected according to actual user requirements. Of course, in the magnetic resonance CEST imaging sequence and apparatus, it is also necessary to cooperate with other necessary hardware or software, which is not repeated here.

The present invention has the following beneficial effects:

The present invention incorporates a lipid multi-peak model into the Bloch-McConnell equation for iterative solving, and fitting is performed on the water, MT, and lipid multi-peak signals. The Bloch-McConnell equation involving lipids may more truly reflect the composition of an acquired signal, thereby obtaining a more accurate CEST reference signal. The lipid artifacts in CEST signals may be eliminated without adjusting the collection sequence, thereby improving the accuracy of CEST imaging.

The aforementioned embodiments are merely implementation modes of the present application and are not intended to limit the present invention. Any changes and improvements made to the present invention without departing from the spirit and range of the present invention, or directly or indirectly applied in other related technical fields, are all included in the present invention, and the protection range is defined by the appended claims.

What is claimed is:

1. A chemical exchange saturation transfer (CEST) lipid artifact elimination method based on a differential analysis with fitted magnetization transfer and lipid signals (DIGITAL), characterized in that CEST raw data acquired by a magnetic resonance imaging apparatus is read through magnetic resonance CEST imaging, lipid artifact elimination and signal extraction are performed on the acquired CEST raw data voxel-wise, and steps of processing each voxel comprise:
Step 1: performing linear-interpolation-based offset correction for a main magnetic field frequency B0 on the acquired CEST raw data to obtain a corrected Z-spectrum corresponding to a target voxel;
Step 2: using data comprising only MT, and data comprising a water direct saturation effect and a lipid direct saturation effect in the corrected Z-spectrum as fitting samples, fitting model parameters of a water-MT-lipid three-pool Bloch-McConnell equation model in respective given upper and lower bounds; and obtaining the water-MT-lipid three-pool Bloch-McConnell equation model by incorporating a lipid multi-peak model into a water-MT two-pool Bloch-McConnell equation;
Step 3: inputting the fitted model parameters into the water-MT-lipid three-pool Bloch-McConnell equation model to generate a CEST reference signal of each frequency point in the corrected Z-spectrum, and subtracting an actual acquired signal in the corrected Z-spectrum from the CEST reference signal corresponding to each frequency point to obtain a CEST effect value of a CEST effect curve corresponding to a frequency point;
Step 4: performing normalization on the CEST effect curve by dividing by (1−FF), wherein FF is a fat fraction obtained by fitting; and in the normalized CEST effect curve, a value of a frequency of interest is the CEST effect value after lipid artifact interference is eliminated.

2. The CEST lipid artifact elimination method based on the DIGITAL according to claim 1, characterized in that the water-MT-lipid three-pool Bloch-McConnell equation model is as follows:
$${\tilde{M}}_{ref} ( t + Δ t ) = exp ( {\tilde{A}}_{w} Δ t ) {\tilde{M}}_{w} ( t ) \cdot ( 1 - FF ) + exp ( {\tilde{A}}_{f} Δ t ) {\tilde{M}}_{f} ( t ) \cdot FF$$
wherein exp represents an exponential function on a basis of a natural constant e; Δt represents an increment of time t; \tilde{M}_{ref}(t+Δt) is total water-lipid magnetization vector as a reference signal at time t+Δt; \tilde{M}_{w}(t) and \tilde{M}_{f}(t) represent a magnetization vector \tilde{M}_{w} of water and a magnetization vector \tilde{M}_{f} of lipid at time t respectively;
$${\tilde{M}}_{w} = ( \begin{matrix}{M}_{w} \\ 1\end{matrix} ) ‵ {\tilde{M}}_{f} = ( \begin{matrix}{M}_{f} \\ 1\end{matrix} ) ;$$
in a matrix M_{w}=[M_{xw}, M_{yw}, M_{zw}, M_{zm}]^{T}, M_{xw}, M_{yw}, M_{zw}, and M_{zm} represent an x-direction magnetization component of a water pool, a y-direction magnetization component of the water pool, a z-direction magnetization component of water group, and the z-direction magnetization component of MT group, respectively; in a matrix M_{f}=[M_{xf1}, M_{yf1}, M_{zf1}, M_{xf2}, M_{yf2}, M_{zf2}, . . . , M_{xf7}, M_{yf7}, M_{zf7}]^{T}, M_{xf1}, M_{xf2}, . . . , and M_{xf7} represent the x-direction magnetization components of 1st to 7th lipid groups respectively, M_{yf1}, M_{yf2}, . . . , and M_{yf7} represent the y-direction magnetization components of the 1st to 7th lipid groups respectively, and M_{zf1}, M_{zf2}, . . . , and M_{zf7} represent the z-direction magnetization components of the 1st to 7th lipid groups respectively; the fat fraction FF=M_{0f}/(M_{0f}+M_{0w}) represents a proportion of a lipid signal in a total water-lipid signal, wherein M_{0f} is a sum of the magnetization components M_{0f1}, M_{0f2}, . . . , and M_{0f7} of the 1st to 7th lipid groups under a steady state, and M_{0w} is the magnetization component of the water group under the steady state; a coefficient matrix
$${\tilde{A}}_{w} = ( \begin{matrix}{A}_{w} & {C}_{w} \\ 0 & 0\end{matrix} ) , {\tilde{A}}_{f} = ( \begin{matrix}{A}_{f} & {C}_{f} \\ 0 & 0\end{matrix} ) ,$$
wherein a matrix
$$\begin{matrix}{A}_{w} = \\ ( \begin{matrix}- {R}_{2 w} & - Δ {ω}_{w} & 0 & 0 \\ {Δω}_{w} & - {R}_{2 w} & - {ω}_{1} & 0 \\ 0 & {ω}_{1} & - {R}_{1 w} - {k}_{wm} & {k}_{mw} \\ 0 & 0 & {k}_{wm} & - {R}_{1 m} - {R}_{rfm} - {k}_{mw}\end{matrix} )\end{matrix} ,$$
and a vector C_{w}=[0,0, R_{1w}M_{0w}, R₁ₘM₀ₘ]^{T}; Δω_{w}=ω_{RF}−ω_{w}ΔB₀, ω_{RF} represents frequency of an applied radio frequency pulse, ω_{w} represents a resonance frequency of the water group, ΔB₀ represents a residual main magnetic field inhomogeneity to be corrected, ω₁ represents an intensity of the applied radio frequency pulse, k_{wm} and kₘw represent exchange rates from the water group to the MT group and from the MT group to the water group, R_{rfm} represents an absorption rate of the MT group to the radio frequency pulse, R_{1w}=1/T_{1w}, R_{2w}=1/T_{2w}, R₁ₘ=1/T₁ₘ, R₂ₘ=1/T₂ₘ represent a longitudinal relaxation rate of the water group, a transverse relaxation rate of the water group, a longitudinal relaxation rate of the MT group, and a transverse relaxation rate of the MT group respectively; a matrix
$${A}_{f} = ( \begin{matrix}{A}_{f 1} & & & \\ & {A}_{f 2} & & \\ & & ⋱ & \\ & & & {A}_{f 7}\end{matrix} ) ,$$
and a vector C_{f}=[0, 0, R_{1f1}M_{0f1}, 0, 0, R_{1f2}M_{0f2}, . . . , 0, 0, R_{1f7}M_{0f7}]^{T}, where an nth matrix
$$\begin{matrix}{A}_{f} = \\ {A}_{f} = ( \begin{matrix}- {R}_{2 fn} & - Δ {ω}_{fn} & 0 \\ {Δω}_{fn} & - {R}_{2 fn} & - {ω}_{1} \\ 0 & {ω}_{1} & - {R}_{1 fn}\end{matrix} )\end{matrix}$$
of a matrix A_{f1}˜A_{f7}, n=1, 2, . . . , to 7; relative amplitude values M_{0f1}, M_{0f2}, . . . , and M_{0f7} of the seven lipid groups are estimated by three parameters comprising CL, ndb, and nmidb, the CL represents fatty acid chain length, the ndb represents a number of double bonds per molecule, and the nmidb represents a number of methylene double bonds; Δω_{fn}=ω_{RF}−ω_{fn}−ΔB₀, wherein the ω_{fn} represents a resonance frequency of an nth group; R_{1fn}=1/T_{1fn} and R_{2fn}=1/T_{2fn} represent a longitudinal relaxation rate and a transverse relaxation rate of the nth lipid group respectively; longitudinal relaxation times of the seven lipid groups are set to be identical: T_{1f}=T_{1f1}=T_{1f2}= . . . =T_{1f7}, a setting of transverse relaxation times of the seven lipid groups is the same: T_{2f}=T_{2f1}=T_{2f2}= . . . =T_{2f7};
the parameters to be fitted in the model are: T_{1w}, T_{2w}, T₂ₘ, M₀ₘ, k_{wm}, FF, T_{1f}, T_{2f}, ΔB₀, CL, ndb, nmidb.

3. The CEST lipid artifact elimination method based on DIGITAL according to claim 2, characterized in that, preferably, the longitudinal relaxation rate R₁ₘ of the MT group and a resonance frequency of lipid peaks are both input as preset values of prior conditions into the model; preferably, the intensity of the applied radio frequency pulse ω₁ is calculated from additionally acquired radio frequency field maps and then input as a prior condition into the model.

4. The CEST lipid artifact elimination method based on DIGITAL according to claim 1, characterized in that, in the Step 2, the data comprising only MT are data points with frequencies preferably in a range from 80 ppm to 6 ppm in the corrected Z-spectrum; the data comprising the water direct saturation effect are data points with frequencies preferably in a range from 1.5 ppm to −1.5 ppm in the corrected Z-spectrum; the data comprising the lipid direct saturation effect are data points with frequencies preferably in a range from −1 ppm to −6 ppm in the corrected Z-spectrum;
further preferably, the data used as fitting samples are data points with frequencies in the ranges from 80 ppm to 10 ppm and 0.75 ppm to −6 ppm in the corrected Z-spectrum.

5. The CEST lipid artifact elimination method based on DIGITAL according to claim 2, characterized in that, preferably, in the Step 2, the parameters to be fitted in the model T_{1w}, T_{2w}, T₂ₘ, M₀ₘ, k_{wm}, FF, T_{1f}, T_{2f}, ΔB₀, CL, ndb, nmidb have upper bounds of [1.50, 0.100, 5e-6, 0.4500, 40, 1, 0.400, 0.150, 64, 18, 5.5, 2.5] respectively, lower bounds of [1.35, 0.015, 1e-6, 0.0001, 20, 0, 0.250, 0.025, −64, 17, 1, 0] respectively during fitting, and initial values [1.35, 0.015, 1e-6, 0.0001, 20, 1, 0.400, 0.025, 0, 17, 1, 0] respectively for fitting;
preferably, in the Step 2, the data in the range from 80 ppm to 10 ppm is set with a higher weight during fitting, and the weight is 2 times a weight of the data in the range from 0.75 ppm to −6 ppm.

6. The CEST lipid artifact elimination method based on DIGITAL according to claim 2, characterized in that, in the Step 2, the absorption rate of the MT group to the radio frequency pulse is R_{rfb}=ω₁²πg(2πω_{w}); wherein g(⋅) is a lineshape function, comprising Gaussian, Lorentzian, and super-Lorentzian; and the lineshape function is preferably super-Lorentzian in breast data.

7. The CEST lipid artifact elimination method based on DIGITAL according to claim 1, characterized in that, in the Step 4, the frequency of interest is 3.5 ppm, and the CEST effect value of 3.5 ppm of the normalized CEST effect curve is an APT^{#} value.

8. A computer-readable storage medium, characterized in that a computer program is stored on the storage medium, and when the computer program is executed by a processor, the CEST lipid artifact elimination method based on DIGITAL according to claim 1 is implemented.

9. A computer-readable storage medium, characterized in that a computer program is stored on the storage medium, and when the computer program is executed by a processor, the CEST lipid artifact elimination method based on DIGITAL according to claim 2 is implemented.

10. A computer-readable storage medium, characterized in that a computer program is stored on the storage medium, and when the computer program is executed by a processor, the CEST lipid artifact elimination method based on DIGITAL according to claim 3 is implemented.

11. A computer-readable storage medium, characterized in that a computer program is stored on the storage medium, and when the computer program is executed by a processor, the CEST lipid artifact elimination method based on DIGITAL according to claim 4 is implemented.

12. A computer-readable storage medium, characterized in that a computer program is stored on the storage medium, and when the computer program is executed by a processor, the CEST lipid artifact elimination method based on DIGITAL according to claim 5 is implemented.

13. A computer-readable storage medium, characterized in that a computer program is stored on the storage medium, and when the computer program is executed by a processor, the CEST lipid artifact elimination method based on DIGITAL according to claim 6 is implemented.

14. A computer-readable storage medium, characterized in that a computer program is stored on the storage medium, and when the computer program is executed by a processor, the CEST lipid artifact elimination method based on DIGITAL according to claim 7 is implemented.

15. A computer electronic device, characterized in comprising a processor and a storage medium, wherein a computer program is stored on the storage medium, and when the computer program is executed by the processor, the CEST lipid artifact elimination method based on DIGITAL according to claim 1 is implemented.

16. A computer electronic device, characterized in comprising a processor and a storage medium, wherein a computer program is stored on the storage medium, and when the computer program is executed by the processor, the CEST lipid artifact elimination method based on DIGITAL according to claim 2 is implemented.

17. A computer electronic device, characterized in comprising a processor and a storage medium, wherein a computer program is stored on the storage medium, and when the computer program is executed by the processor, the CEST lipid artifact elimination method based on DIGITAL according to claim 3 is implemented.

18. A computer electronic device, characterized in comprising a processor and a storage medium, wherein a computer program is stored on the storage medium, and when the computer program is executed by the processor, the CEST lipid artifact elimination method based on DIGITAL according to claim 4 is implemented.

19. A computer electronic device, characterized in comprising a processor and a storage medium, wherein a computer program is stored on the storage medium, and when the computer program is executed by the processor, the CEST lipid artifact elimination method based on DIGITAL according to claim 5 is implemented.

20. A magnetic resonance imaging apparatus configured to eliminate CEST lipid artifacts, characterized in comprising a magnetic resonance scanner and a control unit, wherein the magnetic resonance scanner is configured to obtain a CEST image through magnetic resonance CEST imaging; the control unit obtains the CEST image and stores a computer program, and when the computer program is executed for implementing the CEST lipid artifact elimination method based on DIGITAL according to claim 1, the CEST effect value with lipid artifacts eliminated for each voxel is output.
